(19) United States
(12) Patent Application Publication
(10) Pub. No.: US 2010/0069498 A1
(43) Pub. Date: Mar. 18, 2010

(54) USE OF ALPHA-KETOGLUTARIC ACID FOR THE TREATMENT OF HIGH PLASMA GLUCOSE CONDITION

(75) Inventors: Stefan G. PIERZYNOWSKI, Malmo (SE); Douglas Burrin, Houston, TX (US)

Correspondence Address: MORRISON & FOERSTER LLP, 425 MARKET STREET, SAN FRANCISCO, CA, 94105-2482, US

(73) Assignee: ESSENTYS AB, Lund (SE)

(21) Appl. No.: 12/626,549

(22) Filed: Nov. 25, 2009

Related U.S. Application Data
(62) Division of application No. 10/562,953, filed on Dec. 30, 2005.
(60) Provisional application No. 60/481,301, filed on Aug. 28, 2003.

(30) Foreign Application Priority Data
Jul. 1, 2003 (SE) 0301947-8

(51) Int. Cl. A61K 31/194; C07C 59/347; A61P 3/00

(52) U.S. Cl. 514/574; 562/578

(57) ABSTRACT

A method for improving adsorption of amino acids in a vertebrate, including mammal and bird, is included. The method comprises administering to a vertebrate, including mammal and bird, in a sufficient amount and/or at a sufficient rate to enable a desired effect, AKG, AKG derivates or metabolites, AKG analogues or mixtures thereof. Also contemplated is a method for decreasing adsorption of glucose in a vertebrate, including mammal and bird, comprising administering to the vertebrate, including mammal and bird, in the need thereof, AKG, AKG derivates or metabolites, AKG analogues or mixtures thereof, for decreasing glucose adsorption as well as compositions for use in treatment.

### CROSS REFERENCE TO RELATED APPLICATIONS

This application is a Divisional application of U.S. application Ser. No. 10/562,953, filed Dec. 30, 2005, which is a National Stage application of PCT/SE2004/01062, filed Jul. 1, 2004, which claims the benefit under 35 USC 119(e) to U.S. Provisional Application Ser. No. 60/481,301, filed on Aug. 28, 2003, and claims priority to Sweden Application Serial No. 0301947-8, filed Jul. 1, 2003, all of which are hereby incorporated by reference in the present disclosure in their entirety.

### TECHNICAL FIELD

This invention relates to a method for improving absorption of amino acids as well as a method for decreasing absorption of glucose in a vertebrate, including mammal and bird. Also contemplated is the manufacture of a composition for the improvement of amino acid absorption in said vertebrate.

### BACKGROUND OF THE INVENTION

Diabetes mellitus is a serious metabolic disease that is defined by the presence of chronically elevated levels of plasma glucose. Classic symptoms of diabetes mellitus in adults are polyuria, polydipsia, ketonuria, rapid weight loss together with elevated levels of plasma glucose.

Normal fasting plasma glucose concentrations are less than 115 milligrams per deciliter. In diabetic patients, fasting concentrations are found to be over 140 milligrams per deciliter. In general, diabetes mellitus develops in response to damage to the beta cells of the pancreas. This damage can result from primary diabetes mellitus, in which the beta cells are destroyed by the autoimmune system, or as a secondary diabetic response to other primary diseases, such as pancreatic disease, hormonal abnormalities other than lack of insulin action, drug or chemical induction, insulin receptor abnormalities, genetic syndromes or others.

Primary diabetes mellitus can be classified as Type I diabetes (also called insulin-dependent diabetes ellitus or IDDM) and Type II diabetes mellitus (also called non-insulin dependent diabetes mellitus or NIDDM).

Type I, juvenile onset or insulin-dependent, diabetes is a well-known hormone deficient state, in which the pancreatic beta cells appear to have been destroyed by the body's own immune defence mechanisms. Patients with Type I diabetes mellitus have little or no endogenous insulin secretory capacity. These patients develop extreme hyperglycemia. Type I diabetes was fatal until the introduction of insulin replacement therapy some 70 years ago—first using insulins from animal sources, and more recently, using human insulin made by recombinant DNA technology. It is now clear that the destruction of beta cells in Type I diabetes leads to a combined deficiency of two hormones, insulin and amylin. When pancreatic cells are destroyed, the capacity to secrete insulin and amylin is lost.

The nature of the lesion of the pancreatic beta cells in Type II diabetes is not clear. Unlike the pancreatic beta cells in Type I diabetics, the beta cells of Type II diabetics retain the ability to synthesize and secrete insulin and amylin. Type II diabetes is characterized by insulin and resistance, i.e., a failure of the normal metabolic response of peripheral tissues to the action of insulin. In other words, insulin resistance is a condition where the circulating insulin produces a subnormal biological response. In clinical terms, insulin resistance is present when normal or elevated plasma glucose levels persist in the face of normal or elevated levels of insulin. The hyperglycemia associated with Type II diabetes can sometimes be reversed or ameliorated by diet or weight loss sufficient to restore the sensitivity of the peripheral tissues to insulin. Indeed, Type II diabetes mellitus is often characterized by hyperglycemia in the presence of higher than normal levels of plasma insulin. Progression of Type II diabetes mellitus is associated with increasing concentrations of plasma glucose and coupled with a relative decrease in the rate of glucose-induced insulin secretion. Thus, for example, in late-stage Type II diabetes mellitus, there may be an insulin deficiency.

### Known Treatments and Prophylax of Diabetes Mellitus

The primary aim of treatment in all forms of diabetes mellitus is the same, namely the reduction of plasma glucose concentrations to as near normal as possible, thereby minimizing both the short- and long-term complications of the disease (Tchobroutsky, Diabetologia 15:143-152 (1978)).

The linkage between the extent of hyperglycemia in diabetics and the ensuing long-term complications was further confirmed in the recently completed Diabetes Control and Complications Trial (DCCT) undertaken by the National Institutes of Health (The Diabetes Control and Complications Trial Research Group, N. Eng. J. Med. 329:977 (1993)). The DCCT was conducted over a 10-year period at 29 clinical centres around the United States and Canada, and showed that lowering mean plasma glucose concentrations in Type I diabetics reduced end-organ complications. The development of retinopathy was reduced by 76%, the progression of retinopathy by 54%, and there was an amelioration of the markers of renal disease (proteinuria, albuminuria). The development of significant neuropathic changes was also reduced.

The treatment of Type I diabetes necessarily involves the administration of replacement doses of insulin, administered by the parenteral route. In combination with the correct diet and self-plasma glucose monitoring, the majority of Type I diabetics can achieve a certain level of control of plasma glucose.

In contrast to Type I diabetes, treatment of Type II diabetes frequently does not require the use of insulin. Institution of therapy in Type II diabetes usually involves a trial of dietary therapy and lifestyle modification, typically for 6-12 weeks in the first instance.

Features of a diabetic diet include an adequate but not excessive total calorie intake, with regular meals, restriction of the content of saturated fat, a concomitant increase in the polyunsaturated fatty acid content, and an increased intake of dietary fiber.

Lifestyle modifications include the maintenance of regular exercise, as an aid both to weight control and also to reduce the degree of insulin resistance.

If after an adequate trial of diet and lifestyle modifications, fasting hyperglycemia persists, then a diagnosis of “primary diet failure” may be made, and either a trial of oral hypoglycemic therapy or direct institution of insulin therapy will be required to produce plasma glucose control and, thereby, to minimize the complications of the disease. Type II diabetics who fail to respond to diet and weight loss may respond to therapy with oral hypoglycemic agents such as sulfonylureas or biguanides. Insulin therapy, however, is used to treat other patients with Type II diabetes, especially those who have undergone primary dietary failure and are not obese, or those who have undergone both primary diet failure and secondary oral hypoglycemic failure.

The use of amylin agonists in the treatment of diabetes mellitus has been described in U.S. Pat. Nos. 5,124,314 and 5,175,145. Excess amylin action mimics key features of Type II diabetes and amylin blockade has been proposed as a novel therapeutic strategy.

Known treatments are e.g. diabetes pills based on e.g. Sulfonylureas that help pancreas to make more insulin and help the body to use the insulin better. Possible side effects: hypoglycemia, upset stomach, skin rash or itching and weight gain.

Other pills are based on biguanides that restricts glucose production by the liver, and also lowers the amount of insulin in the body, improve blood fat and cholesterol. Possible side effects are sickness in combination with alcohol, worsening of existing kidney and problems, weakness, dizziness trouble to breath, nausea, and diarrhoea.

Other pills are based on alpha-glucosidase inhibitors block the enzymes that digest starch. Possible side effects are stomach problems.

Other pills are based on thiazolidinediones that helps the cells to become more sensitive to insulin. Possible side effects are that they are not to be used in combination with liver disease (regularly check-ups), hypoglycemi, and only in combination with other treatment, less effective birth control by pills, gain of weight, anemia risk, swelling (edema).

Other pills are based on meglitinides that helps the pancreas to make more insulin after meals. Possible side effects are hypoglycemia, and weight gain.

Further, combination oral medicines exists, based on e.g. glyburide (sulfonylurease) and metformin (biguanide) named e.g. “Glucovance”. Possible side effects are hypoglycemia, not to be used together with kidney disease, and should not be used in combination with alcohol.

U.S. Pat. No. 5,234,906 discloses compositions comprising glucagon and an amylin agonist and their use to control or treat hyperglycemic conditions.

WO 93/10146 discloses amylin agonists and their use to treat or prevent hypoglycemic conditions including insulin-requiring states such as diabetes mellitus.

### Renal Failure and Malnutrition

Renal failure or renal malfunction is the state when kidneys fail to clean the blood from waste products. The kidney failure causes an accumulation of the toxic waste products in the blood. The kidneys normally have excess cleaning capacity and the renal capacity could be 50% of normal before symptoms occur. Symptoms are itching, tiredness, nausea, vomiting, loss of appetite leading to malnutrition. Renal failure is often associated with diabetes and high blood pressure. The symptoms mentioned above, i.e. vomiting and loss of appetite leads to malnutrition in a subject suffering from renal failure.

The dialysis procedure will reduce the pressure from the waste products on the kidneys. Still, it is a time consuming procedure, which the patient may need to perform several times a week. The patient undergoing a dialysis procedure needs medical attention and the procedure is both costly and time consuming

### Glutamate Oxidation

Since the in situ rat studies of Windmueller and Spaeth (1), it has been known that glutamate and glutamine are important metabolic fuels for the small intestine Windmueller and Spaeth were the first to report the large fractional metabolism of glutamate (≈95%) and glutamine (≈70%) by the intestinal tract during absorption. Their results have since been confirmed in vivo in both piglets (2) and humans (3).

During glutamate oxidation, the first step is transamination by any number of enzymes, deamination by glutamate dehydrogenase (GDH), many of which are expressed in the gastrointestinal tract (4,5). Deamination by GDH yields AKG and free ammonia. During transamination by branched-chain amino transferase (BCAT), glutamate donates an amino moiety to a branched-chain α-keto-acid, forming AKG and the corresponding branched-chain amino acid.

### Alpha-Ketoglutaric Acid

Glutamine and its derivatives e.g., alpha-ketoglutaric acid (AKG) are molecules which have a central role in the systemic and gut metabolism via Krebs cycle. However, the mechanisms are still not fully understood (Pierzynowski, S. G., and Sjödin, A. (1998) J. Anim. a. Feed Sci. 7: 79-91; and Pierzynowski S. G., et al. Eds: K B K Knutsen and J-E Lindberg., Uppsala 19-21 Jun. 2001).

AKG (2-oxo-pentanedioic acid, 2-oxoglutaric acid, alpha-oxoglutaric acid, alpha-oxopentanedioic acid, 2-Ketoglutaric acid, 2-oxo-1,5-pentanedioic acid, 2-oxopentanedioic acid, 2-oxo-glutaric acid) can theoretically be a product of glutamine, glutamate, glutamic acid degradation in body metabolism. It may also serve as a precursor not only for glutamine and arginine, but also for some other amino acids, and are thus regarded as a protein catabolic protector. Olin at al., 1992 showed that when AKG was added to fish feed, urea emission was reduced. Similarly, in humans where AKG is added to total parentheral nutrition (TPN) solutions mixed with others amino acids, good protection of nitrogen loss after surgery is observed (Pierzynowski, S. G., and Sjödin, A. (1998) J. Anim. a. Feed Sci. 7: 79-91). In the case of humans, the AKG is probably integrated with muscle protein degradation to serve requirements of the intestinal tract during so-called postoperative stress, e.g. catabolism, starvation, etc.

The requirements of glutamine family related metabolites for gut function was proven recently by Reeds et al., (1996, Am. J. of Physiol.—Endocrinology and Metabolism 270:413-418) who reported almost 100% glutamate/glutamine utilisation in the first pass in infant pigs small intestine.

AKG can be an important energy donor via few transformation pathways e.g. via ornithine and putrescine to GABA or succinate. Theoretically, AKG can also work as an ammonium ion scavenger possibly via transformation to glutamate/glutamine.

It is recognised—but has never been published—that enterocytes are dependent on their growth from ammonium.

It is thus highly desirable in the light of the aforementioned problems to develop means and methods for treating and preventing hypoglycemic conditions, such as diabetes mellitus, as well as malnutrition often associated with diabetes and e.g. renal failure, in mammals, e.g. cats, dogs or humans, where problems, or side effects associated with prior art means and methods may be avoided. Also, there is a need for increasing the well being as in addition to the nutritional status in renal patients, as well as diabetic patients. In this respect, the present invention addresses those needs and interests.

### SUMMARY OF THE INVENTION

In view of the foregoing disadvantages known in the art of preventing, treating and/or alleviating diabetes as well as other hypoglycemic related diseases and the high medical costs for doing so, and for correcting malnutrition associated with e.g. diabetes and renal failure, the present invention provides new and improved methods and compositions for preventing, treating and/or alleviating diabetes and malnutrition.

An object of the present invention is to provide a method for improving absorption of amino acids in a vertebrate, including mammal and bird. The method comprises administering, to a vertebrate, including mammal and bird, in a sufficient amount and/or at a sufficient rate to enable a desired effect on amino acid absorption, AKG, AKG derivates or metabolites, AKG analogues, or mixtures thereof.

One embodiment of the method is wherein the AKG, AKG derivates or metabolites, AKG analogues or mixtures thereof are selected from the group consisting of alpha-ketoglutaric acid (AKG), ornitine-AKG, arginine-AKG, glutamine-AKG, glutamate-AKG, leucine-AKG, chitosan-AKG and other salts of AKG with amino acids and amino acids derivates; mono- and di-metal salts of AKG such as CaAKG, Ca(AKG)₂, and NaAKG.

A further embodiment is wherein the vertebrate is a rodent, such as a mouse, rat, guinea pig, or a rabbit; a bird, such as a turkey, hen, chicken or other broilers; farm animals, such as a cow, a horse, a pig, piglet or other free going farm animals; or a pet, such as a dog, or a cat.

One further embodiment is wherein the vertebrate is a human being.

Yet another embodiment is wherein the amino acid is any essential amino acid.

A further embodiment is wherein the essential amino acid is isoleucin, leucin, lysine, and proline.

The invention further comprises a method for decreasing absorption of glucose in a vertebrate, including mammal and bird. The method comprises administering, to a vertebrate, including mammal and bird, in a sufficient amount and/or at a sufficient rate to enable a desired effect on glucose absorption, AKG, AKG derivates or metabolites, AKG analogues, or mixtures thereof.

The invention further comprises a method for preventing, inhibiting, or alleviating a high glucose condition in a vertebrate, including mammal and bird. The method comprises administering to a vertebrate, including mammal and bird, in a sufficient amount and/or at a sufficient rate to enable a desired effect on said condition, AKG, AKG derivates or metabolites, AKG analogues, or mixtures thereof.

One embodiment is wherein the high glucose condition is Type I or Type II diabetes mellitus.

The invention further comprises use of AKG, AKG derivates or metabolites, AKG analogues, or mixtures thereof, for the manufacture of a composition for the prevention, alleviation or treatment of a high glucose condition.

One embodiment is wherein the high glucose condition is diabetes mellitus type I or II.

The invention also relates to the use of AKG, AKG derivates or metabolites, AKG analogues or mixtures thereof, for the manufacture of a composition for the prevention, alleviation or treatment of malnutrition.

One embodiment of the use is wherein the composition is a pharmaceutical composition with optionally a pharmaceutically acceptable carrier and/or additives.

Another embodiment of the use is wherein the composition is a food or a feed supplement.

Yet a further embodiment is wherein the food or feed supplement is a dietary supplement and/or a component in the form of solid food and/or beverage.

One further embodiment is wherein the AKG, AKG derivates or metabolites, AKG analogues or mixtures thereof, in the manufactured composition is in a therapeutically effective amount.

One further embodiment is wherein the therapeutically effective amount is 0.01-0.2 g/kg bodyweight per daily dose.

### SHORT DESCRIPTION OF DRAWINGS

FIG. 1 shows whole-body leucine kinetics in control and AKG infused pigs. Values are mean±SEM; n=9, each pig received both control and AKG. Values for AKG were not different from control using analysis of variance (ANOVA). AKG, α-ketoglutarate; NOLD, non-oxidative leucine disposal; Ra, leucine appearance rate; Balance, Ra subtracted from NOLD represents protein body leucine balance.

### DETAILED DESCRIPTION OF THE INVENTION

### Definitions

In the context of the present application and invention the following definitions apply:

The term “pharmaceutical composition” as used herein, refers to a therapeutically effective composition according to the invention.

The term “therapeutically effective amount”, or “effective amount”, or “therapeutically effective”, as used herein, refers to that amount which provides a therapeutic effect for a given condition and administration regimen. This is a predetermined quantity of active material calculated to produce a desired therapeutic effect in association with the required additive and diluent; i.e., a carrier, or administration vehicle. Further, it is intended to mean an amount sufficient to reduce and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent; i.e., carrier, or additive. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art.

The term “derviate” or “derivative” is herein intended to mean a chemical substance derived from mother substance either directly or by modification or partial substitution.

The term “analogue” or analog” is herein intended to mean compounds that are structurally similar to another, but are not necessarily isomers. Analogs have similar function(s) but differ in structure or evolutionary origin.

As used herein, “treating”, refers to treating with a goal towards curing, which may be a full/complete or a partial curing of a condition or conditions.

The term “alleviate” is herein intended to mean not only a reduction of intensity of a condition or indication, but also postponing onset of a condition or indication.

The term “prevent” is herein intended to mean ensuring that something does not happen, e.g. that a condition or indication relating to an immature GIT does not happen. By preventing a certain condition or indication, the onset of such condition or indication is postponed.

The term “increased amino acid absorption” is herein intended to mean a change in the net absorption of amino acids in a vertebrate compared to a vertebrate, not obtaining treatment or administration according to the invention. The changes are regarded as increased if the net absorption is quantitatively larger in said vertebrate as compared to a vertebrate of the same species not obtaining said treatment.

The term “kinetics” is herein intended to mean a continuous or frequent monitoring or measurement of the readings in absorption of amino acids as well as glucose in a vertebrate to determine its absorption rate.

The term “sodium-AKG” as used herein is used interchangeably with the terms “AKG-Na”, “Na-AKG”, “Na salt of AKG”, “AKG (Na salt)”.

The term “chitosan-AKG” as used herein is used interchangeably with the terms “AKG-chitosan”, “AKG (chitosan salt)”.

### Diagnosis of Diabetes Type I and Type II

Diagnosis of patients afflicted with Type I and Type II diabetes is well within the ability and knowledge of one skilled in the art. For example, individuals over the age of 35 who have symptoms of polydipsia, polyuria, polyphagia (with or without weight loss) coupled with elevated plasma glucose concentrations and without a history of ketoacidosis are generally considered within the diagnosis of Type II diabetes mellitus. The presence of obesity, a positive family history for Type II diabetes and normal or elevated fasting plasma insulin and c-peptide concentrations are additional characteristics of most patients with Type II diabetes mellitus. By “therapeutically effective amount” is meant an amount, either in single or multiple doses, which beneficially reduces plasma glucose concentrations in a subject afflicted with Type II diabetes mellitus.

The inventors have now surprisingly found that the site of infusion had an effect on AKG adsorption. After a duodenally infused AKG, an increased absorption of amino acids and a decreased absorption of glucose were surprisingly observed.

The present invention may thus be used to lower plasma glucose in a non-insulin-taking Type II diabetic subject.

### Diagnosis of Malnutrition

Diagnosis of patients afflicted with malnutrition, i.e. with a faulty or inadequate nutrition intake or undernourishment, is well within the ability and knowledge of one skilled in the art. Normally a general health status of the individual is performed to assess malnutrition.

### Diagnosis of Renal Failure

Diagnosis of patients afflicted with renal failure is well within the ability and knowledge of one skilled in the art.

There are two forms of renal failure, acute and chronic renal failure (ACF and CRF). Acute renal failure can normally be reversed, while chronic renal failure normally progresses. CRF treatment is divided into pre-dialysis and active treatment of uremia using e.g. dialysis or transplantation. There exists no exact definition of pre-dialysis as to the starting point but normally pre-dialysis is defined as the period in time between the diagnosis of renal failure and the initiation of active treatment. Dialysis and transplantation is considered as active treatment.

### A Method for Improving Absorption of Amino Acids

According to the invention, a method for improving absorption of amino acids in a vertebrate, including mammal and bird is disclosed. The method comprises administering, to a vertebrate, including mammal and bird, in a sufficient amount and/or at a sufficient rate to enable a desired effect on amino acid absorption, AKG, AKG derivates or metabolites, AKG analogues or mixtures thereof.

The amino acid absorption is considered improved when compared to amino acid absorption in a vertebrate, including mammal and bird, not obtaining said AKG, AKG derivates or metabolites, AKG analogues or mixtures thereof.

Further embodiments of the method are wherein the AKG, AKG derivates or metabolites, AKG analogues or mixtures thereof are selected from the group consisting of alpha-ketoglutaric acid (AKG), ornitine-AKG, arginine-AKG, glutamine-AKG, glutamate-AKG, leucine-AKG, chitosan-AKG and other salts of AKG with amino acids and amino acids derivates; mono- and di-metal salts of AKG such as CaAKG, CaAKG₂, and NaAKG.

Further embodiments are wherein the vertebrate is a rodent, such as a mouse, rat, guinea pig, or a rabbit; a bird, such as a turkey, hen, chicken or other broilers; farm animals, such as a cow, a horse, a pig, piglet or free going farm animals; or a pet, such as a dog, or a cat.

A further embodiment is wherein the vertebrate is a human being. The human being may be a patient in the need of treatment of malnutrition due to e.g. renal failure, diabetes mellitus, athletes, age (children and elderly), pregnancy, anorexia nervosa, bulimia nervosa, Binge eating disorder, compulsive overeating, or other eating disorders not otherwise specified (EDNOS).

The vertebrate, such as said human, may in further embodiments be any vertebrate in the need of increasing the availability and utilisation of amino acids, e.g. essential amino acids, or conditionally amino acids, particularly isoleucine, leucine, lysine, and proline.

Examples of essentially amino acids are alpha-amino acids, such as isoleucinde (Ileu), leucine (Leu), lysine (Lys), methionine (Met), phenylalanine (Phe), threonine (Thr), tryptophan (Try), and valinine (Val) in humans. Essential amino acids differ from species to species. Rats need two other amino acids, namely arginine (Arg) and histidine (His).

Further embodiments are wherein the amino acid is any amino acid such as alanine, valine, leucine, isoleucine, proline, phenylalanine, tryptophan, methionine, threonine, cysteine, tyrosine, glutamine, histidine, lysine, arginine, aspartate, asparagine, glutamate, glutamine, glycine, and serine.

Further embodiments are wherein the amino acid is any essential, or conditionally essential, amino acid. Examples of essential, or conditionally essential, amino acids are given in table 2.

A further embodiment is wherein the essential, or conditionally essential, amino acid is selected from the group consisting of isoleucin, leucin, lysine, and proline.

### A Method for Decreasing Absorption of Glucose and a Method for Preventing, Inhibiting, or Alleviating an Increase in Plasma Glucose

Plasma glucose level is the amount of glucose (sugar) in the blood. It is also known as serum glucose level. The amount of glucose in the blood is expressed as millimoles per litre (mmol/l) or mg/dL.

Normally, plasma glucose levels stay within narrow limits throughout the day, about 4 to 8 mmol/l in a human being. The glucose levels are higher after meals and usually lowest in the morning. Fasting levels are normally about 70-110 mg/dL (3.9-6.1 mmol/L) and 2 hours after a meal the levels normally are about 80-140 mg/dL (4.4-7.8 mmol/L). A plasma glucose level of >180 mg/dL (>10.0 mmol/l) 2 hours after a meal is normally considered a high plasma glucose value. This is also the case when having a plasma glucose value of >140 mg/dL when fasting.

If a person has e.g. diabetes, their plasma glucose level sometimes moves outside these limits. The basic defect in all patients with diabetes is the decreased ability of insulin to induce cells of the body to remove glucose (sugar) molecules from the blood. Whether this decreased insulin activity is due to a decreased amount of insulin produced (e.g. Type I Diabetes), or from the insensitivity of the cells to a normal amount of insulin, the results are the same, i.e. plasma glucose levels which are too high. This is termed “hyperglycemia”, or “hyperglycaemia”, or “hyperglucemi” which means “high glucose in the blood”. Normally, hyperglycemia is when the plasma glucose is >240 mg/dL (>13.4 mmol/L).

According to the invention, a method for decreasing absorption of plasma glucose in a vertebrate, including mammal and bird, is disclosed. The method comprises administering to a vertebrate, including mammal and bird, in a sufficient amount and/or at a sufficient rate to enable a desired effect on glucose absorption AKG, AKG derivates or metabolites, AKG analogues or mixtures thereof.

The decrease after administration of AKG, AKG derivates or metabolites, AKG analogues or mixtures thereof in glucose absorption may be 5-50%, such as 5, 10, 15, 20, 25, 30, 35, 40, 45, or 50% of the starting plasma glucose value.

In a further embodiment, the decrease in absorption is 20-40% of the starting plasma glucose value.

In a further embodiment, the decrease is 30% of the starting plasma glucose value.

Further, a method for preventing, inhibiting, or alleviating a high plasma glucose condition in a vertebrate, including mammal and bird, is disclosed. Said method comprises administering, to a vertebrate, including mammal and bird, in a sufficient amount and/or at a sufficient rate to enable a desired effect said high glucose condition, AKG, AKG derivates or metabolites, AKG analogues, or mixtures thereof.

In a further embodiment, the high glucose condition is a hyperglycemic condition.

Said methods relating to a high glucose or a hypoglucemic conditions include further embodiments wherein the AKG, AKG derivates or metabolites, AKG analogues or mixtures thereof, are selected from the group consisting of alpha-ketoglutaric acid (AKG), ornitine-AKG, arginine-AKG, glutamine-AKG, glutamate-AKG, leucine-AKG, chitosan-AKG and other salts of AKG with amino acids and amino acids derivates; mono- and di-metal salts of AKG such as CaAKG, CaAKG₂, and NaAKG.

Even further embodiments are wherein the vertebrate is a rodent, such as a mouse, rat, guinea pig, or a rabbit; a bird, such as a turkey, hen, chicken or other broilers; farm animals, such as a cow, a horse, a pig, piglet or free going farm animals; or a pet, such as a dog, or a cat.

Even further embodiments are wherein the vertebrate is a human being.

Even further embodiments are wherein said high glucose conditions are due to e.g. Acromegaly, Cushing's syndrome, Hyperthyroidism, Pancreatic cancer, Pancreatitis, Pheochromocytoma, insufficient amount of insulin, or excessive food intake

Even further embodiments are wherein the high glucose levels are due to Type I or Type II diabetes mellitus.

### Use of AKG for Diabetes Mellitus and for Treatment of Malnutrition

According to the invention use of AKG, AKG derivates or metabolites, AKG analogues or mixtures thereof for the manufacture of a composition for the prevention, alleviation or treatment of a high glucose condition is disclosed.

Examples of high glucose conditions and hyperglycemic conditions have been given in the preceding paragraph.

Further embodiments include wherein the hyperglycemic condition is diabetes mellitus type I or II.

According to the invention use of AKG, AKG derivates or metabolites, AKG analogues, or mixtures thereof, for the manufacture of a composition for the prevention, alleviation or treatment of malnutrition, is disclosed.

In further embodiments of said uses said composition is a pharmaceutical composition with optionally a pharmaceutically acceptable carrier and/or additives.

In further embodiments the composition is a food or a feed supplement.

In further embodiments, the food or feed supplement is a dietary supplement and/or a component in the form of solid food and/or beverage.

In further embodiments, the AKG, AKG derivates or metabolites, AKG analogues or mixtures thereof, in the manufactured composition is in a therapeutically effective amount.

In further embodiments the therapeutically effective amount is 0.01-0.2 g/kg bodyweight per daily dose.

### Administration of AKG, AKG Derivates or Metabolites, AKG Analogues or Mixtures Thereof.

According to the methods disclosed above, AKG, AKG derivates or metabolites, AKG analogues or mixtures thereof, is administered to a vertebrate, including mammal and bird; a rodent, such as a mouse, rat, guinea pig, or a rabbit; a bird, such as a turkey, hen, chicken or other broilers; farm animals, such as a cow, a horse, a pig, piglet or free going farm animals; or a pet, such as a dog, or a cat.

Administration may be performed in different ways depending on what species of vertebrate is to be treated, the condition of the vertebrate in need of said methods, and the specific indication to treat.

In one embodiment, the administration is performed as a food or feed supplement, such as a dietary supplement and/or a component in form of solid food and/or beverage. Further embodiments may be in the form of suspensions or solutions, such as a beverage further described below.

Also, the dosage forms may include capsules or tablets, such as chewable or soluble, e.g. effervescent tablets, as well as powder and other dry formats known to the skilled man in the art, such as pellets, such as micropellets, granules and grains.

The administration may be in the form of parenteral, rectal or oral food or feed supplement, as revealed above. Parenteral vehicles include sodium chloride solution, Ringer's dextrose, dextrose and sodium chloride, lactated Ringer's or fixed oils.

The food and feed supplement may also be emulsified. The active therapeutic ingredient may then be mixed with excipients, which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH, buffering agents, which enhance the effectiveness of the active ingredient.

Different formats of the parenteral food or feed supplement may be supplied, such as solid food, liquids or lyophilized or otherwise dried formulations. It may include diluents of various buffers (e.g., Tris-HCl., acetate, phosphate), pH and ionic strength, additives such as albumin or gelatine to prevent absorption to surfaces, detergents (e.g., Tween 20, Tween80, Pluronic F68, bile acid salts), solubilizing agents (e.g., glycerol, polyethyleneglycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the composition, complexation with metal ions, or incorporation of the material into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts.

### A Beverage

In one embodiment, the food or feed supplement is administered in the form of a beverage, or a dry composition thereof, in any of the methods according to the invention.

The beverage comprises an effective amount of AKG, AKG derivates or metabolites, AKG analogues or mixtures thereof together with a nutritionally acceptable water-soluble carrier, such as minerals, vitamins, carbohydrates, fat and proteins. Examples of AKG, AKG derivates or metabolites, AKG analogues or mixtures thereof are alpha-ketoglutaric acid (AKG), ornitine-AKG, arginine-AKG, glutamine-AKG, glutamate-AKG, leucine-AKG, chitosan-AKG and other salts of AKG with amino acids and amino acids derivates; mono- and di-metal salts of AKG such as CaAKG, CaAKG₂, and NaAKG.

All of these components are supplied in a dried form if the beverage is provided in a dry form. A beverage provided ready for consumption further comprises water. The final beverage solution may also have a controlled tonicity and acidity, e.g. as a buffered solution according to the general suggestions in the paragraph above.

The pH is preferably in the range of about 2-5, and in particularly about 2-4, to prevent bacterial and fungal growth. A sterilised beverage may also be used, with a pH of about 6-8.

The beverage may be supplied alone or in combination with one or more therapeutically effective composition(s).

### Use of AKG, AKG Derivates or Metabolites, AKG Analogues or Mixtures Thereof.

According to the invention, a use of AKG, AKG derivates or metabolites, AKG analogues or mixtures thereof, is disclosed for the manufacture of a composition for the prevention, alleviation or treatment of hyperglycemic conditions, such as diabetes type I and Type II, as well as for treatment of malnutrition.

Further embodiments of the invention include a use, wherein the composition is a pharmaceutical composition. This pharmaceutical composition may be together with a pharmaceutically acceptable carrier and/or additives, such as diluents, preservatives, solubilizers, emulsifiers, adjuvants and/or carriers useful in the methods and use disclosed in the present invention.

Further, as used herein “pharmaceutically acceptable carriers” are well known to those skilled in the art and may include, but are not limited to, 0.01-0.05M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Even further embodiments of the invention include a use, wherein the composition is a dietary supplement and/or a component in the form of solid food and/or beverage.

Such a manufactured composition, such as a pharmaceutical composition or a food or feed supply, comprises a composition according to the invention, and may optionally comprise a carrier and/or an amount of a second or further active ingredient affecting any hyperglycemic condition, such as diabetes Type I and II, as well as malnutrition.

### Dose of the Administered Pharmaceutical Composition

According to the invention, the use of AKG, AKG derivates or metabolites, AKG analogues or mixtures thereof, for the manufacture of a composition according to the invention includes an administration of a therapeutical effective amount to the vertebrate, such as a bird or mammal in the need thereof. Such a therapeutically effective amount is about 0.01-0.2 g/kg bodyweight per daily dose.

### AKG, AKG Derivates or Metabolites, AKG Analogues or Mixtures Thereof.

According to the invention, of AKG, AKG derivates or metabolites, AKG analogues or mixtures thereof are included. Example of AKG, AKG derivates or metabolites, AKG analogues or mixtures thereof are alpha-ketoglutaric acid (AKG), ornitine-AKG, arginine-AKG, glutamine-AKG, glutamate-AKG, leucine-AKG, chitosan-AKG, and other salts of AKG with amino acids and amino acids derivates; mono- and di-metal salts of AKG such as CaAKG, CaAKG₂ NaAKG.

### Administration Targets

As can be readily appreciated by one of ordinary skill in the art, the methods and pharmaceutical compositions of the present invention are particularly suited for administration to any vertebrate in the need thereof, such as a bird, including but not limited to, a turkey, hen or chicken and other broilers and free going animals, or a mammal, including but not limited to, domestic animals, such as feline or canine subjects, farm animals, such as, but not limited to, bovine, equine, caprine, ovine, and porcine subjects, wild animals, whether in the wild or in a zoological garden, research animals, such as mice, rats, rabbits, goats, sheep, pigs, dogs, cats, etc., i.e. for veterinary medical use.

Also, human beings are included as administration targets in the treatment of any high glucose levels or hyperglycemic condition, such as diabetes Type I and Type II, as well as any condition associated with malnutrition, after e.g. renal failure, diabetes Type I and Type II.

Further, the administration targets may also be any vertebrate, such as the one mentioned above, in the need of increasing the availability and utilisation of amino acids, e.g. essential amino acids, or conditionally amino acids, particularly isoleucine, leucine, lysine, and proline. The human being may also be a patient in the need of treatment of malnutrition or of increasing the availability and utilisation of amino acids due to e.g. renal failure, surgical treatments, e.g. pancreatectomy or transplantation, geriatric conditions, diabetes mellitus, athletes, age (children and elderly), pregnancy, anorexia nervosa, bulimia nervosa, Binge eating disorder, compulsive overeating, nutritional disorders, metabolic disturbances, or other eating disorders not otherwise specified (EDNOS), bedsores, vertebrates having no appetite, or due to a wasting disease.

### REFERENCES

(All references cited herein are hereby incorporated by reference in their entirety).
1. Windmueller, H. G., & Spaeth, A. E. (1975) Intestinal metabolism of glutamine and glutamate from the lumen as compared to glutamine from blood. Arch. Biochem. Biophys. 171: 662-672.
2. Stoll, B., Burrin, D. G., Henry, J., Hung, Y., Jahoor, F., & Reeds, P. J. (1999) Substrate oxidation by the portal drained viscera of fed piglets. Am. J. Physiol. 277: E168-E175.
3. Matthews, D. E., Marano, M. A., & Campbell, R. G. (1993) Splanchnic bed utilization of glutamine and glutamic acid in humans. Am. J. Physiol. 264: E848-E854.
4. Madej, M., Lundh, T., & Lindberg, J. E. (1999) Activities of enzymes involved in glutamine metabolism in connection with energy production in the gastrointestinal tract epithelium of newborn, suckling and weaned piglets. Biol. Neonate 75: 250-258.
5. Suryawan, A., Hawes, J. W., Harris, R. A., Shimomura, Y., Jenkins, A. E., & Hutsun, S. M. (1998) A molecular model of human branched-chain amino acid metabolism. Am. J. Clin. Nutr. 68: 72-81.
6. Lambert, B. D., Stoll, B., Niinikoski, H., Pierzynowski, S., & Burrin, D. G. (2002) Net portal absorption of enterally fed alpha-ketoglutarate is limited in young pigs. J. Nutr. 132:3383-3386.
7. Kristensen, N. B., Jungvid, H., Fernandez, J. A., & Pierzynowski, S. G. (2002) Absorption and metabolism of α-ketoglutarate in growing pigs. J. Anim. Physiol. Anim. Nutr. 86:239-245.
8. Bergmeyer, H. U., & Bernt, E. (1974) 2-oxoglutarate. UV spectrophotometric determination. In: Methods of enzymatic analysis, 2nd Ed. (Bergmeyer, H. U., ed.). Academic Press, New York, N.Y.
9. Pajor, A. M. (1999) Sodium-coupled transporters for krebs cycle intermediates. Annu Rev. Physiol. 61:663-682.
10. Murphy, J. M., Murch, J. M., and Ball, R. O. (1996) Proline is synthesized from glutamate during intragastric infusion but not during intravenous infusion in neonatal piglets. J. Nutr. 126:878-886.

### EXAMPLES

The invention is illustrated below by a number of non-limiting examples. While the invention has been described in relation to certain disclosed embodiments, the skilled person may foresee other embodiments, variations or combinations which are not specifically mentioned but are nonetheless within the scope of the appended claims.

### Materials and Methods Section for Example 1-2

### Animal Maintenance

Housing and care of the animals conformed to the U.S. Department of Agriculture guidelines.

### Study Design

Female piglets (n=9) were purchased from the Texas Department of Criminal Justice, Huntsville, Tex.

Pigs (14 d of age) arrived at the Children's Nutrition Research Center and for a 7-day adjustment period were fed a liquid milk replacer diet (Litter Life, Merrick, Middleton, Wis.) at a rate of 50 g/(kg·d).

The composition of the milk replacer (per kg dry matter) was 500 g lactose, 100 g fat, and 250 g protein.

After 7 days, food was withdrawn from the piglets overnight and they were prepared for surgery as described previously (2).

Briefly, under isoflurane anesthesia and aseptic conditions, the piglets were implanted with a polyethylene catheter (o.d., 1.27 mm, Becton Dickinson, Sparks, Md.) in the common portal vein, and silastic catheters (o.d., 1.78 mm) in an external jugular vein and a carotid artery.

An ultrasonic flow probe (8 to 10 mm i.d., Transonic, Ithaca, N.Y.) was placed around the portal vein.

A silicone catheter (o.d., 2.17 mm, Baxter Healthcare, McGaw Park, Ill.) was implanted into the lumen of the duodenum. The catheters were filled with sterile saline containing heparin (2.5×10⁴ U/L), and exteriorized on either the left flank (portal and duodenal catheters, flow probe leads) or between the scapulae (jugular and carotid catheters).

Immediately preoperatively, animals received an intramuscular injection of antibiotic (20 mg/kg enrofloxacin, Bayer, Shawnee Mission, Kans.) and an intramuscular injection of analgesic (0.1 mg/kg butorphenol tartrate, Fort Dodge Labs, Fort Dodge, Iowa).

Before enteral feeding was resumed postoperatively, pigs were maintained on total parenteral nutrition for 24 h at a rate of 5 mL·kg⁻¹·h⁻¹. Pigs were allowed 7 d to recover from surgery. In all piglets, intakes and rates of weight gain had returned to pre-operative levels.

### Preparation of Samples

Blood samples were immediately placed on ice and centrifuged.

Plasma was collected, immediately frozen in liquid N₂, and stored at −80° C. until analysis.

### Amino Acid Analysis

For plasma amino acid analysis, a 0.2 mL aliquot of plasma was mixed with an equal volume of an aqueous solution of methionine sulfone (4 mmol/L) and centrifuged at 10,000×g for 120 min through a 10-kDa cutoff filter.

A 50 μL aliquot of the filtrate was dried and the amino acids were analyzed by reverse-phase HPLC of their phenylisothiocyanate derivatives (Pico Tag, Waters, Woburn, Mass.).

Plasma AKG was determined by the method of Bergmeyer and Bernt (8) with minor modifications.

Briefly, the assay was carried out in 0.5 mL of working solution consisting of 100 mmol/L phosphate buffer (pH 7.6), 4 mmol/L ammonium chloride, and 50 μmol/L NADH.

To the working solution, an appropriate amount of plasma containing 1-10 nmol of AKG was added.

An initial absorbance reading was obtained at 340 nm.

Following the initial absorbance reading, ˜6 units (in a volume of 10 μL) of bovine GDH (G2501; Sigma-Aldrich, St. Louis, Mo.) was added to each tube.

After a 10-minute incubation, a second absorbance reading was taken at 340 nm.

The amount of AKG in the sample is directly proportional to the decrease in absorbance between the first and second reading.

The AKG concentration was calculated by the use of a standard curve.

### Plasma Ammonia Determination

Plasma ammonia was determined using a spectrophotometric assay kit (171-C, Sigma-Aldrich, St. Louis, Mo.).

### Plasma Glucose Determination

Plasma glucose was determined using a spectrophotometric assay kit (315-100; Sigma-Aldrich, St. Louis, Mo.).

### Blood Bicarbonate Determination

To estimate the enrichment of blood bicarbonate, an aliquot of whole blood (1.0 mL) was placed in a 10-mL Vacutainer (Becton Dickinson, Franklin Lakes, N.J.), and 0.5 mL of perchloric acid (10% wt/wt) was added.

Room air (10 mL) filtered through soda lime (Sodasorb; Grace Container Products, Lexington, Mass.) was injected into the Vacutainer, removed into a gas-tight syringe, and transferred to a second Vacutainer.

The isotopic enrichment of the carbon dioxide in the gas sample was measured on a continuous flow gas isotope ratio mass spectrometer (ANCA; Europa Instruments, Crewe, U.K.).

### Determination of Plasma Ketoisocaproic Acid

Plasma ketoisocaproic acid (KIC) was isolated by cation exchange chromatography (AG-50V resin, Bio-Rad).

Eluants were treated with sodium hydroxide (100 μL; 10 N) and hydroxylamine HCl (200 μL; 0.36 M) and heated (60° C.; 30 min) After cooling, the pH of the samples was adjusted to <2.

The keto acids were extracted in 5 mL of ethylacetate and dried under nitrogen at room temperature.

Derivitization of KIC was accomplished by adding 50 μL of N-methyl-N-t-butyl-dimethylsilys-trifluoroacetamide+1% t-butyl-dimethylchlorosilane.

The isotopic enrichment of KIC was determined by EI GC-MS (Hewlett Packard 5970 GC-mass spectrometer with a Hewlett Packard 5890 Series II GC) by monitoring ions at 316 m/z and 317 m/z.

### Determination of Plasma Urea Isotopic Enrichment

Plasma urea isotopic enrichments were determined by EI GC-MS analysis. Proteins were precipitated from 50 μL of plasma with 200 μL of ice-cold acetone.

After vortexing, the protein was separated by centrifugation, and the supernatant was removed and dried under nitrogen.

To the dried supernatant, 250 μL of a 1:20 dilution of malonaldehyde bid(dimethyl acetal) and concentrated HCl (30 wt %) was added, the sample was incubated at room temperature for 2 h, and then evaporated to dryness (Speedvac, Savant Instruments, Form a Scientific, Marietta, Ohio).

The urea was derivitized with 50 μL of N-methyl-N-t-butyl-dimethylsilys-trifluoroacetamide+1% t-butyl-dimethylchlorosilane and the isotopic enrichment in plasma was determined using EI GC-MS analysis by monitoring ions at 153 to 155 m/z.

### Calculations

Net portal balance of metabolites [μmol/(kg·h)] was calculated as follows:

(Conc._{PORT}−Conc._{ART})×PBF (1)

when Conc. is the concentration in the blood (μmol/L), PORT and ART refer to portal and arterial blood and PBF is portal blood flow [L/(kg·h)].

Whole body leucine flux [Q; μmol/(kg·h)], was calculated as follows:

Q=R*[(IE_{infusate}/IEₚₗₐₛₘₐ)−1] (2)

where R is the tracer infusion rate [μmol/(kg·h)] and IE_{infusate} and IEₚₗₐₛₘₐ are the isotopic enrichments (expressed as mol %) of the infused tracer and plasma KIC, respectively.

Body CO₂ production was calculated as follows:

$$Body {CO}_{2} production = \frac{\begin{matrix}( \frac{IE infusate}{IE arterial bicarbonate} - 1 ) \times \\ tracer infusion rate\end{matrix}}{0.82} \qquad ( 3 )$$

where IE infusate is the enrichment of H¹³CO₃⁻ in the infusate (mole percentage excess), IE arterial bicarbonate is the enrichment in arterial blood (mole percentage excess), and tracer infusion rate [μmol/(kg·h)] during the i.v. bicarbonate infusion which proceeded each treatment period. The entire equation was divided by 0.82 to correct for recovery of infused labelled carbon in bicarbonate.

Whole-body leucine oxidation [μmol/(kg·h)] was calculated as follows:

[IE_{CO2}/IE_{KIC}]×Equation 3 (4)

where IE_{CO2} is the isotopic enrichment of bicarbonate during the 1-¹³C-leucine infusion and IE_{LEU} is the isotopic enrichment of 1-¹³C-KIC during the 1-¹³C-leucine infusion.

Whole-body non-oxidative leucine disposal (NOLD) is an estimate of leucine incorporation into muscle. NOLD [μmol/(kg·h)] was calculated by the following equation:

NOLD=Equation 2−Equation 4 (5)

Whole-body leucine appearance rate (Ra) [μmol/(kg·h)] is an estimate of protein catabolism and was calculated as:

Ra=Equation 2−leucine intake (6)

Whole-body urea flux was calculated as follows:

Urea flux=[([¹⁵N₂] urea IE/[¹⁵N₂] urea PE)−1]×[¹⁵N₂] urea IR (7)

where IE is infusate enrichment, PE is plasma enrichment at steady state during urea infusion and IR is infusion rate.

### Statistical Analysis

For all statistical tests, a p-value of 0.05 was considered to represent statistical significance.

In Exp. 1, the effects of AKG on the arterial, portal, and net portal appearance of individual amino acids, AKG, glucose, ammonia, and leucine kinetics were analyzed using the General Linear Model procedure (Minitab. Inc., State College, Pa.). The model contained the effects of AKG supplementation and pig. Pig was included as a random variable. Treatment means were computed using the LSMEANS option. A one-way Student's T test was used to test if AKG net portal balance was significantly greater than zero during control treatments.

### Example 1

### Measurements of Plasma AKG, Glucose, Ammonia, Blood Flow and Whole Body Urea Flux

### Objective

The objective of this example is to evaluate the effect of AKG infusion on plasma AKG, glucose, ammonia, blood flow and whole body urea flux.

### Animal Experiments

Piglets were deprived of food for 15 h before initiation of the experiment.

On the day of the experiment, at time −1 h, a primed (7.75 mL/kg; 25% wt/wt aqueous solution; oral), continuous duodenal infusion of milk replacer [Litter Life, Merrick, Middleton, Wis.; 7.75 mL/(kg·h)] prepared as a 25% (wt/wt) aqueous solution which provided ˜920 kJ and 12.5 g protein/(kg·d).

Either saline (control; 930 mmol/L NaCl) or sodium-AKG (Na-AKG), 930 mmol/L, from Sigma-Aldrich, St. Louis, Mo.) were dissolved in the milk replacer.

The level of AKG was chosen based on previous data (6) from out laboratory, where intakes of greater than 2.5% of diet dry matter was required to observe a detectable portal balance of AKG.

Pigs also received an intravenous (200 μmol/kg), continuous, 6-h infusion of ¹⁵N₂-urea [20 μmol/(kg·h)] (98%; Cambridge Isotope Laboratories).

At time 0 h, a primed (15 μmol/kg), continuous, 2-h infusion of NaH¹³CO₂ (15 μmol/(kg·h); 99%; Cambridge Isotope Laboratories, Andover, Mass.) was initiated.

Arterial samples were obtained at 0, 90, 105, and 120 minutes after initiation of NaH¹³CO₂ infusion to determine whole body CO₂ production.

At time 2 h, the NaH¹³CO₂ infusion was terminated and a primed (40 μmol/kg), continuous, 4-h infusion of 1-¹³C-Leucine (40 μmol/(kg·h); 99%; Cambridge Isotope Laboratories) was initiated.

Arterial and portal venous samples were obtained at time 4, 5, and 6 h for determination of leucine and urea kinetics as well as mass balance of ammonia, AKG, glucose, and amino acids.

All pigs received both control and AKG treatment in a completely randomized design with at least 24 h between treatment periods.

### Results

Plasma AKG, glucose, ammonia, blood flow, and whole body urea flux is presented in Table 1.

TABLE 1
Effects of AKG infusion on metabolite concentration, net portal
balance, and whole body 1-¹³C-leucine, and ¹⁵N₂-urea kinetics.

| | AKG¹ (% of diet dry matter) | | |
|---|---|---|---|
| | 0 | 3.75 | P |
| AKG infusion rate, | 0 | 930 | — |
| μmol/(kg · h) | | | |
| Portal blood flow, | 3.21 ± 0.28² | 3.36 ± 0.27 | 0.34 |
| L/(kg · h) | | | |
| Arterial AKG, μmol/L | 13.8 ± 1.7 | 27.4 ± 3.6 | <0.01 |
| Portal AKG, μmol/L | 22.0 ± 1.4 | 64.6 ± 5.9 | <0.001 |
| AKG net portal balance, | 19.7 ± 2.8 | 95.2 ± 12 | <0.001 |
| μmol/(kg · h) | | | |
| AKG net portal balance, | — | 10.23 ± 0.57 | — |
| % of infused | | | |
| Glucose net portal balance, | 303.1 ± 61 | 203.9 ± 69 | <0.05 |
| mg/(kg · h) | | | |
| Ammonia net portal balance, | 520.1 ± 66 | 561.1 ± 53 | 0.91 |
| μmol/(kg · h) | | | |
| Whole body urea flux, | 398.3 ± 35 | 377.8 ± 39 | 0.56 |
| μmol/(kg · h) | | | |

¹AKG, α-ketoglutarate;
²SEM

AKG infusion [930 μmol/(kg·h)] increased (P<0.01) arterial and portal AKG concentration and the net portal balance of AKG. Even when no AKG was infused into the duodenum, the net portal absorption of AKG [19.7±2.8 μmol/(kg·h)] was significantly greater than 0. However, the net portal absorption of AKG was increased (P<0.001) with AKG treatment when compared to control. The net portal balance of AKG was 95 μmol/(kg·h) which represented only 10.23% of the amount infused.

The net portal balance of 10.23% is actually a slight overestimate of infused AKG absorption because when only saline was infused, there was a statistically significant absorption of AKG. If a correction is made for absorption of AKG from the control diet, the proportion of infused AKG appearing in the portal venous drainage decreases to 8.12%.

Interestingly, net portal balance of glucose was decreased (P<0.05) with AKG treatment. Portal blood flow, ammonia net portal balance and whole body urea flux were not affected by AKG treatment.

Both the arterial and portal concentrations of proline were increased (P<0.05) and portal leucine tended (P<0.01) to be increased by AKG treatment (data not shown). The portal mass balance of amino acids is presented in Table 2. AKG treatment increased (P<0.05) the portal mass balance of leucine, lysine and proline, and tended (P<0.10) to increase the portal mass balance of isoleucine.

TABLE 2
Net portal balances of amino acids in pigs receiving a duodenal
infusion of 0 or 930 μmol/(kg · h) AKG (n = 5).

| | Control | | AKG¹ | |
|---|---|---|---|---|
| Amino acid | $\frac{Portal balance}{μmol \text{/} ( kg \cdot h )}$ | % of Intake | $\frac{Portal balance}{μmol \text{/} ( kg \cdot h )}$ | % of Intake |

Essential amino acids

| | | | | |
|---|---|---|---|---|
| Isoleucine | 164.5 ± 26 | 100.1 | 230.2^{b} ± 28 | 140.0 |
| Leucine | 294.9 ± 44 | 76.3 | 438.6^{a} ± 50 | 113.4 |
| Phenylalanine | 80.4 ± 11 | 83.3 | 95.2 ± 11 | 98.7 |
| Valine | 218.5 ± 33 | 85.2 | 279.3 ± 32 | 108.9 |
| Histidine | 27.7 ± 11 | 43.1 | 45.9 ± 3.8 | 71.4 |
| Threonine | 185.0 ± 40 | 66.4 | 210.9 ± 18 | 75.7 |
| Lysine | 237.7 ± 35 | 72.3 | 324.5^{a} ± 37 | 98.8 |
| Tryptophan | 38.6 ± 6.4 | — | 47.2 ± 4.3 | — |

Conditionally essential amino acids

| | | | | |
|---|---|---|---|---|
| Arginine | 95.2 ± 24 | 85.8 | 109.0 ± 19 | 98.3 |
| Proline | 216.4 ± 25 | 69.9 | 354.5^{a} ± 32 | 114.5 |
| Tyrosine | 85.7 ± 12 | 100.6 | 115.8 ± 17 | 135.9 |

Nonessential amino acids

| | | | | |
|---|---|---|---|---|
| Alanine | 539.6 ± 61 | 182.9 | 557.8 ± 48 | 189.0 |
| Aspartate | 28.2 ± 4.6 | 9.2 | 29.7 ± 6.0 | 9.6 |
| Asparagine | 169.9 ± 23 | — | 185.6 ± 18 | — |
| Glutamate | 64.2 ± 23 | 14.9 | 80.1 ± 17 | 18.6 |
| Glutamine | 17.2 ± 12 | — | 25.5 ± 45 | — |
| Glycine | 167.0 ± 27 | 109.4 | 177.2 ± 20 | 116.0 |
| Serine | 213.3 ± 89 | 94.4 | 244.7 ± 64 | 108.3 |

^{a}Different from control (P ≦ 0.05);
^{b}Different from control (P < 0.10)
¹AKG, α-ketoglutarate;
²LSMean ± SEM

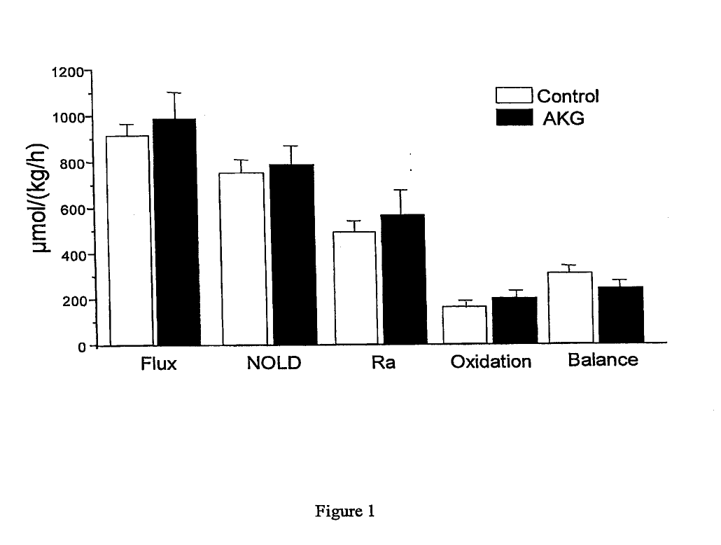

Whole-body leucine kinetics are shown in FIG. 1. Whole body flux, NOLD, Ra, and oxidation were not affected by AKG treatment.

### Example 2

### Measurements of Mean Luminal Disappearance of AKG

### Objective

The objective of this example is to evaluate the mean luminal disappearance of an infused AKG bolus.

### Animal Experiments

Pigs (n=7) were given a duodenal bolus infusion (7.75 mL/kg; 25% (wt/wt) aqueous solution) of liquid milk replacer (Litter Life, Merrick) containing 25 mg/mL sodium AKG (1040 μmol/kg BW).

After 1 h, pigs were sacrificed.

The small intestine was carefully clamped at the proximal duodenum and distal ileum, removed and flushed with 2×50 mL of saline to wash the intestine.

The washes were collected, pooled, and a 15 mL aliquot was flash frozen in liquid N₂ and stored at −80° C. for later AKG analysis.

### Results

An AKG bolus of 1040 μmol/kg was infused. The mean luminal disappearance was 663±38 μmol/kg in one hour. This represents 63.8 of the 1040 μmol/kg of infused AKG.

### Discussion and General Conclusion of Experiment 1 and 2

In Example 1, AKG was continuously infused into the duodenum and only 10% of the infused AKG appeared in the portal venous drainage.

The observation that only 10% of infused AKG appeared in the portal plasma raises several possibilities as to the fate of luminal AKG. One possible explanation for the low AKG portal appearance is that luminal AKG transport is limited. Sodium/dicarboxylate cotransporters, which are capable of transporting AKG, exist on pig brush border membranes (9), so it seems unlikely that AKG would not be taken up by the enterocytes. To test this, we infused a single duodenal bolus of 1040 μmol/kg and found that over 660 μmol/kg disappeared from the small intestine of piglets in 1 h (Example 2). Thus, approximately 64% of the AKG bolus had disappeared from the lumen of the duodenum in only 1 h.

The net portal appearance of glutamate and glutamine were not affected by AKG infusion as observed previously (6). If the absorbed AKG was converted to glutamate, it could either be released into the portal blood, or converted to other amino acids.

It might, however, be expected that the release of glutamate and glutamine would not be increased by AKG even if substantial conversion to these amino acids occurred, given that very little dietary glutamate or glutamine is released by the PDV under normal feeding conditions (ref 1,2). It has been shown (10) that proline can be synthesized from enteral glutamate by the intestinal tissue. Given that the increase in proline net portal balance was 138.1 μmol/(kg·h) in AKG treated pigs, and that over 800 μmol/(kg·h) of AKG was unaccounted for in the portal balance, it is possible that the increase in proline net portal balance can be completely accounted for by conversion from AKG. However, such a large conversion of AKG to proline in the enterocyte, should have led to a decrease in portal ammonia balance, but portal ammonia balance remained unchanged. The lack of effect on portal ammonia balance was also reflected similar rates of whole body urea synthesis in the two groups.

Branched chain amino acid (BCAA) transaminase catalyzes the reaction between AKG and branched chain amino acids (leucine, isoleucine, and valine). The BCAA is transaminated, forming glutamate from AKG and the respective keto-acid from each of the BCAA. Supplemental AKG may lead to a decrease in the net release of BCAA from the PDV by stimulating the transamination of BCAA to form glutamate. However, the portal release of the leucine was increased by AKG, yet this did not affect whole body leucine kinetics. The net portal balance of lysine was also increased with AKG. Because the net-portal balance of many amino acids was near 100% for many amino acids with the AKG treatment, it is not clear if AKG spared the amino acids or increased amino acid release due to proteolysis within the portal-drained viscera.

An additional probable fate of AKG within the enterocyte is oxidation via the TCA cycle. If indeed all of the carbon infused as AKG was oxidized to CO₂, an increase CO₂ output from the PDV would be expected, yet the production of CO₂ in the whole body did not increase with AKG infusion. Interestingly, the net portal balance of glucose was decreased with AKG treatment.

Because substantial quantities of AKG disappeared from the lumen of the small intestine, but cannot be accounted for in the portal venous drainage in either AKG, or in net balance of amino acid products of AKG metabolism, the fate of enterally fed AKG remains unclear. However, when AKG was infused into the duodenum, only 10% of the luminal supply appeared in the portal venous drainage, yet this amount of AKG was sufficient to increase portal balance and circuiting concentration of the compound. Thus, despite the uncertainty regarding the precise metabolic fate of AKG in the lumen, the results indicate that the intestinal availability of dietary AKG is limited.

The resulting increase in circulating AKG had no effect on the net portal appearances of glutamate, glutamine, ammonia, BCAA.

Additionally, increased systemic AKG had no effect on PDV or whole body leucine kinetics or urea flux. These results agree with previous data where AKG was provided intragastrically.

### Example 3

### Comparative Influence of Na-AKG and Chitosan-AKG Administered Enterally, on Amino Acid and Keto Acid Resorption to the Enterocytes and Blood Plasma, and their Metabolism

### Objective

The objective of this example is to compare the influence of Na-AKG (or Na salt of AKG), and chitosan-AKG administered enterally, on amino acid and keto acid resorption to the enterocytes and blood plasma and their metabolism. Also, the influence of Na-AKG and chitosan-AKG on keto acid transformation to amino acid is measured by monitoring blood plasma amino acids level. This study will test the hypothesis that AKG influences enteral transformation of keto acids to amino acids and improves protein synthesis.

### Animal Experiments

A total three pigs was used in this experiment; these pigs had a body weight of approximately 20 kg. The pigs were separated in boxes and fed on a standard diet for 4-5 days to adapt to the new facility. Pigs were then surgically implanted with catheters and intestinal cannulas and allowed 3-7 days to recover.

The surgical procedures used were those typically used in the field and known to persons skilled in the art.

After the operation, a 3-day recovery period was allowed in this case and the pigs were fed once a day (at a time of 10.00) with a standard feed (3% body weight). After the recovery period, the amino acid level in blood plasma was measured under the conditions of Na-AKG administration (see experiment (ii)), chitosan-AKG administration (experiment (iii)) and no AKG administration (experiment (i)); control experiment), more details of which are presented below.

### Conditions of AKG Administration:

### Experiment (i):

The keto acids or amino acids (Amines) (a total volume of 50 ml) were infused intraduodenally (i.d.) in a dose of *“morning feed equivalent” for 1 hour. (*“morning feed equivalent” means that the animals obtained approximately the same amount of amino-acids as normally present in food corresponding to the morning feed.)

10 portions were given over 1 h (50 ml dose+50 ml saline)

This represented the control experiment.

Blood samples (at baseline** level, 0 h) and at 1, 2, 4 hours, were taken. (**The baseline sample is defined as the sample at time 0, before amino acids/ketoacids infusion.)

Blood samples (5 ml whole blood for amino acid analysis, from artery, portal, hepatic vein) were collected on ethylenediaminetetraacetic acid (EDTA) with aprotinin in order to stop coagulation and proteinase activity.

(Treatment may involve the use of 5 drops EDTA+trasylol, centrifugation and the plasma frozen at −20° C.)

### Experiment (ii)

The keto acids or amino acids (Amines) mixed with Na-AKG (in a total volume of 50 ml), were infused intraduodenally (i.d.) in a dose of *“morning feed equivalent” for 1 hour. (10 portions were given over 1 h, 50 ml dose, optionally with saline).

Blood samples (at baseline level, 0 h) and at 1, 2, 4 hours, were taken.

Blood samples (5 ml whole blood for amino acids analysis, from artery, portal, hepatic vein) were collected on ethylenediaminetetraacetic acid (EDTA) with aprotinin in order to stop coagulation and proteinase activity.

Experiment (iii)

The keto acids or amino acids (Amines) mixed with chitosan-AKG (in a total volume of 50 ml), were infused intraduodenally (i.d.) in a dose of *“morning feed equivalent” for 1 hour. (10 portions were given over 1 h, 50 ml dose, optionally with saline).

Blood samples (at baseline level, 0 h) and at 1, 2, 4 hours, were taken.

Blood samples (5 ml whole blood for amino acids analysis, from artery, portal, hepatic vein) were collected on ethylenediaminetetraacetic acid (EDTA) with aprotinin in order to stop coagulation and proteinase activity.

### Results

Table 3 below shows the results of this investigation:

TABLE 3
Incremental increase of free amino acids in the blood after administration
of amino-acids:

| | ΔI (mmol/L) | ΔII (mmol/L) | ΔI (mmol/L) | ΔII (mmol/L) |
|---|---|---|---|---|
| Time (hr) | Artery | Artery | Hepatic vein | Hepatic vein |
| 1 | −0.5^{a} | 0.65^{c} | 0.43^{A} | 0.23^{A} |
| 1.5 | 0.04^{b} | 1.11^{d} | 1.61^{B} | 1.85^{BC} |
| 2.5 | −0.48^{a} | 1.69^{d} | 1.59^{B} | 1.94^{C} |

I represents Na-AKG salt
II represents chitosan-AKG salt
Δ increment in time = (amino acids at Δtime 0 − amino acids level at 1, 1.5 and 2.5 h)
The differing small or capital letters given with the results describe statistical differences when p < 0.05.

### Discussion and General Conclusions for Example 3

The example shows that chitosan-AKG salt improves essential amino acid absorption. This improvement is better than that achieved using Na-AKG.

This observation is important and relevant for better utilisation of dietary amino acids for the improvement of amino acid absorption in impaired gut tissue, found, for example, in diabetic or elderly patients.

1. A method for improving absorption of amino acids in a vertebrate, including mammal and bird, the method comprising administering to a vertebrate, including mammal and bird, in a sufficient amount and/or at a sufficient rate to enable a desired effect on amino acid absorption AKG, AKG derivates or metabolites, AKG analogues, or mixtures thereof.

2. The method according to claim 1, wherein the AKG, AKG derivates or metabolites, AKG analogues or mixtures thereof, are selected from the group consisting of alpha-ketoglutaric acid (AKG), ornitine-AKG, arginine-AKG, glutamine-AKG, glutamate-AKG, leucine-AKG, chitosan-AKG, and other salts of AKG with amino acids and amino acid derivates; mono- and di-metal salts of AKG such as CaAKG, Ca(AKG)₂, and NaAKG.

3. The method according to claim 1, wherein the vertebrate is a rodent, such as a mouse, rat, guinea pig, or a rabbit; a bird, such as a turkey, hen, chicken or other broilers; farm animals, such as a cow, a horse, a pig, piglet or free going farm animals; or a pet, such as a dog, or a cat.

4. The method according to claim 1, wherein the vertebrate is a human being.

5. The method according to claim 1, wherein the amino acid is any essential amino acid.

6. The method according to claim 5, wherein the essential amino acid is isoleucine, leucine, lysine, and proline.

7. A method for decreasing absorption of plasma glucose in a vertebrate, including mammal and bird, the method comprising administering to a vertebrate, including mammal and bird, in a sufficient amount and/or at a sufficient rate to enable a desired effect on glucose absorption, AKG, AKG derivates or metabolites, AKG analogues, or mixtures thereof.

8. A method for preventing, inhibiting, or alleviating a high plasma glucose condition in a vertebrate, including mammal and bird, the method comprising administering to a vertebrate, including mammal and bird, in a sufficient amount and/or at a sufficient rate to enable a desired effect on said condition, AKG, AKG derivates or metabolites, AKG analogues, or mixtures thereof.

9. The method according to claim 7, wherein the AKG, AKG derivates or metabolites, AKG analogues or mixtures thereof are selected from the group consisting of alpha-ketoglutaric acid (AKG), ornitine-AKG, arginine-AKG, glutamine-AKG, glutamate-AKG, leucine-AKG, chitosan-AKG and other salts of AKG with amino acids and amino acids derivates; mono- and di-metal salts of AKG such as CaAKG, and NaAKG.

10. The method according to claim 7, wherein the vertebrate is a rodent, such as a mouse, rat, guinea pig, or a rabbit; a bird, such as a turkey, hen, chicken or other broilers; farm animals, such as a cow, a horse, a pig, piglet or free going farm animals; or a pet, such as a dog, or a cat.

11. The method according to claim 7, wherein the vertebrate is a human being.

12. The method according to claim 8, wherein the high plasma glucose condition is Type I or Type II diabetes mellitus.

13. Use of AKG, AKG derivates or metabolites, AKG analogues or mixtures thereof, for the manufacture of a composition for the prevention, alleviation or treatment of a high plasma glucose condition.

14. The use according to claim 13, wherein the high plasma glucose condition is diabetes mellitus type I or II.

15. Use of AKG, AKG derivates or metabolites, AKG analogues or mixtures thereof, for the manufacture of a composition for the prevention, alleviation or treatment of malnutrition.

16. The use according to claim 13, wherein the composition is a pharmaceutical composition with optionally a pharmaceutically acceptable carrier and/or additives.

17. The use according to claim 13, wherein the composition is a food or a feed supplement.

18. The use according to claim 17, wherein the food or feed supplement is a dietary supplement and/or a component in the form of solid food and/or beverage.

19. The use according to claim 13, wherein the AKG, AKG derivates or metabolites, AKG analogues or mixtures thereof, in the manufactured composition, is in a therapeutically effective amount.

20. The use according to claim 19, wherein the therapeutically effective amount is 0.01-0.2 g/kg bodyweight per daily dose.
